(19) United States
(12) Patent Application Publication
(10) Pub. No.: US 2007/0021377 A1
(43) Pub. Date: Jan. 25, 2007

(54) Pharmaceutical composition comprising a macrolide immunomodulator

(76) Inventors: Maximilian Grassberger, Wien (AT); Stefan Hirsch, Lorrach (DE); Friedrich Karl Mayer, Oberwil (CH); Nabila Sekkat, Basel (CH); Anton Stutz, Wien (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/550,356

(22) PCT Filed: Apr. 2, 2004
(86) PCT No.: PCT/EP04/03514

§ 371 Date: Nov. 3, 2005

(30) Foreign Application Priority Data
Apr. 4, 2003 (GB) 0307867.2

(51) Int. Cl. A61K 31/739; A61K 31/395; A61K 31/202

(52) U.S. Cl. 514/54; 514/183; 514/560

(57) ABSTRACT

Snyergistic combination of a macrolide T-cell immunomodulator or immunosuppressant such as 33-epichloro-33-desoxyascomycin and a ceramide such as ceramide 3, LPC-9S or linoleic acid are provided, which are useful in particular in the treatment of dermatological or mucosal diseases such as atopic or contact dermatitis or dry skin, asteatotic eczema or xerosis.

The invention relates to pharmaceutical compositions, for use in particular in the treatment of skin diseases. It concerns a pharmaceutical composition comprising a macrolide T-cell immunomodulator or immunosuppressant and a ceramide.

It has now been found that, surprisingly, macrolide T-cell immunomodulators and immunosuppressants, when used in combination with ceramides, act synergistically, resulting in a potentiation of pharmacological activity, such that effective beneficial, especially anti-dermatitis activity is seen upon co-administration at dosages which would be well below the effective dosages administered individually.

The invention thus concerns novel pharmaceutical compositions comprising a macrolide T-cell immunomodulator or immunosuppressant in association or combination with a ceramide, hereinafter briefly named “the compositions of the invention”.

A macrolide T-cell immunomodulator or immunosuppressant is to be understood herein as being a T-cell immunomodulator or T-cell immunosuppressant which has a macrocyclic compound structure including a lactone or lactam moiety. While it preferably has at least some T-cell immunomodulating or immunosuppressant activity, it may also exhibit concomitantly or predominantly further pharmaceutical properties, such as anti-inflammatory activity.

A ceramide is to be understood herein as being an N-acyl fatty acid derivative of a sphingosine (1,3-dihydroxy-2-amino-4-octadecene), or a derivative thereof, such as a glycosphingolipid, e.g. an N-acyl fatty acid derivative of a sphingosine where the acyl group is derived from a fatty acid of 18 to 26 carbon atoms. It may be a mixture of sphingosine or phytosphingosine derivatives, containing saturated or unsaturated acids, e.g. non-hydroxy-substituted or α-hydroxy- or ω-hydroxy-substituted. The term “ceramide” as used herein includes synthetic analogues of natural ceramides, e.g. as known under the term pseudoceramide.

The natural ceramides represent the most abundant group of stratum corneum lipids. They are structural lipids present in the intercellular spaces of the stratum corneum (J. Invest. Dermat. 87 [1986] 758-761).

The compositions of the invention may be adapted for systemic, e.g. oral or intravenous, or for topical use; preferably they are adapted for topical use. They are useful for the known indications of the particular active agents incorporated therein. They are particularly indicated for use in dermatological or mucosal diseases, e.g. dermatological or mucosal diseases which have an inflammatory component or involve inflammatory complications, such as atopic or contact dermatitis or dry skin, asteatotic eczema and xerosis, and for restorement of the lipid skin barrier in the stratum corneum.

Oral administration of essential unsaturated fatty acids such as linoleic acid has been shown to have a beneficial effect in atopic dermatitis patients, presumably by restoration of functional ceramides (B. Melnik et al., Br. J. Dermatol. 119 [1988) 547-548).

A suitable macrolide T-cell immunomodulator or immunosuppressant is for example an FKBP12-binding calcineurin inhibitor or mitogen-activated kinase modulator or inhibitor, in particular an asco- or rapamycin. It preferably is an ascomycin. While the macrolide preferably has at least some calcineurin- or mitogen-activated kinase modulating or inhibiting activity, it may also exhibit concomitantly or predominantly further pharmaceutical properties, such as antiinflammatory activity. It preferably is a compound, e.g. an ascomycin, having rather long-acting activity relatively to other members of the same structural class, e.g. it is metabolically degraded slowly to inactive products.

An asco- or rapamycin is to be understood as asco- or rapamycin as such, or a derivative thereof. An asco- or rapamycin derivative is to be understood as being an antagonist, agonist or analogue of the parent compound which retains the basic structure and modulates at least one of the biological, for example immunological properties of the parent compound.

An “anti-inflammatory ascomycin derivative” is defined herein as an ascomycin derivative that exhibits pronounced anti-inflammatory activity in e.g. animal models of allergic contact dermatitis but has only low potency in suppressing systemic immune response, namely, which has a minimum effective dose (MED) of up to a concentration of about 0.04% w/v in the murine model of allergic contact dermatitis upon topical administration, while its potency is at least 10 times lower than for tacrolimus (MED 14 mg/kg) in the rat model of allogeneic kidney transplantation upon oral administration (Meingassner, J. G. et al., Br. J. Dermatol. 137 [1997] 568-579; Stuetz, A. Seminars in Cutaneous Medicine and Surgery 20 [2001] 233-241). Such compounds are preferably lipophilic.

Suitable ascomycins are e.g. as described in EP 184162, EP 315978, EP 323042, EP 423714, EP 427680, EP 465426, EP 474126, WO 91/13889, WO 91/19495, EP 484936, EP 523088, EP 532089, EP 569337, EP 626385, WO 93/5059 and WO 97/8182;

in particular:

ascomycin;
tacrolimus (FK506; Prograf®);
imidazolylmethoxyascomycin (WO 97/8182 in Example 1 and as compound of formula I);
32-O-(1-hydroxyethylindol-5-yl)ascomycin (L-732531) (Transplantation 65 [1998] 10-18, 18-26, on page 11, FIG. 1; and
(32-desoxy,32-epi-N1-tetrazolyl)ascomycin (ABT-281) (J. Invest. Dermatol. 12 [1999] 729-738, on page 730, FIG. 1); preferably:
{1R,5Z,9S,12S-[1E-(1R,3R,4R)],13R,14S,17R,18E,21 S,23S,24R,25S,27R}-17-ethyl-1,14-dihydroxy-12-[2-(4-hydroxy-3-methoxycyclohexyl)-1-methylvinyl]-23,25-dimethoxy-13,19,21,27-tetramethyl-11,28-dioxa-4-azatricyclo[22.3.1.0(4,9)]octacos-5,18-diene-2,3,10,16-tetraone (Example 8 in EP 626385),
hereinafter referred to as “5,6-dehydroascomycin”;
{1E-(1R,3R,4R)]1R,4S,5R,6S,9R,10E,13S,15S,16R,17S,19S,20S}-9-ethyl-6,16,20-trihydroxy-4-[2-(4-hydroxy-3-methoxycyclohexyl)-1-methylvinyl]-15,17-dimethoxy-5,11,13,19-tetramethyl-3-oxa-22-azatricyclo[18.6.1.0(1,22)]heptacos-10-ene-2,8,21,27-tetraone (Examples 6d and 71 in EP 569337), hereinafter referred to as “ASD 732”; and especially
pimecrolimus (INN recommended) (ASM981; Elidel™), i.e. {[1E-(1R,3R,4S)]1R,9S,12S, 13R,14S,17R,18E,21S,23S,24R,25S,27R}-12-[2-(4-chloro-3-methoxycyclohexyl)-1-methylvinyl]-17-ethyl-1,14-dihydroxy-23,25-dimethoxy-13,19,21,27-tetramethyl-11,28,dioxa-4-azatricyclo[22.3.1.0(4,9)]octacos-18-ene-2,3,10,16-tetraone, of formula I 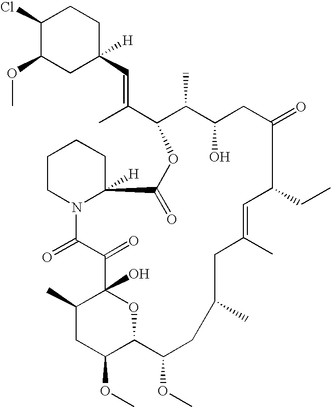
   (Example 66a in EP 427680), hereinafter also referred to as “33-epichloro-33-desoxyascomycin”.

Suitable anti-inflammatory ascomycin derivatives are e.g.: (32-desoxy-32-epi-N-tetrazolyl)ascomycin (ABT-281); 5,6-dehydroascomycin; ASD 732; and pimecrolimus.

Suitable rapamycins are e.g. as described in U.S. Pat. No. 3,929,992, WO 94/9010 and U.S. Pat. No. 5,258,389, preferably sirolimus (rapamycin; Rapamune®) and everolimus (RAD001; Certican°).

A suitable ceramide is for example:
a natural ceramide, e.g. as described in J. Invest. dermatol. 84 (1985) 410-412, e.g. ceramide 3 [M. Kerscher et al., Eur. J. Dermatology 1 [1991] 39-43; S. A. Long et al., Arch. Dermatol. Res. 277 (1985) 284-287];
a pseudoceramide (Eur. J. Dermatology 1 [1991] 39-43; J. Clin. Invest. 94 [1994] 89-96), e.g. A-pseudoceramide, or PC-9S (20th World Congress of Dermatology, Paris [Jul. 1-5, 2002], BookII, Poster Abstracts, Abstr. 228, p. 1S, 415);
a ceramide-based barrier repair agent such as TriCeram® [which contains 2.1% ceramides, 0.8% free fatty acids and 0.8% cholesterol by weight in an oil-in-water vehicle comprising lanolin];
   or another agent that contains ceramide or pseudoceramide or that influences ceramide homeostase, e.g. an agent that stimulates ceramide synthesis, such as a ceramide precursor, e.g. an essential unsaturated fatty acid such as linoleic acid, or that inhibits ceramide degradation by e.g. ceramidases (ceramidase inhibitor); preferably ceramide 3, PC-9S or linoleic acid.

Subgroups of compositions of the invention comprise a macrolide T-cell immunomodulator or immunosuppressant, preferably an anti-inflammatory ascomycin derivative as defined above, especially pimecrolimus, in combination or association with a ceramide other than the following ceramides singly or collectively in any number:
a sphingolipid; and/or
a C₁₂-24 fatty acid; and/or
a ceramidase inhibitor; and/or
a glucosylceramide; and/or
ceramide-3.

In a further subgroup of compositions of the invention the macrolide T-cell immunomodulator or immunosuppressant is other than tacrolimus. In a further subgroup it is other than tacrolimus and sirolimus. In a further subgroup it is other than tacrolimus, sirolimus and ascomycin.

A particularly preferred composition of the invention is pimecrolimus in association or combination with ceramide-3.

Preferred for use in the treatment of conditions where inflammation is involved are compositions of the invention wherein one or both components possess some degree of inherent anti-inflammatory activity. Particularly preferred are compositions comprising an ascomycin in combination with a ceramide, especially 33-epichloro-33-desoxyascomycin in combination with ceramide 3, PC-9S or linoleic acid. The inflammatory condition is e.g. atopic or contact dermatitis or dry skin, asteatotic eczema or xerosis.

“Treatment” as used herein includes prevention, namely prophylactic as well as curative treatment.

Synergy is e.g. calculated as described in Berenbaum, Clin. Exp. Immunol. 28 (1977) 1, using an interaction term to correct for differences in mechanism between the two drugs, as described in Chou et al., Transpl. Proc. 26 (1994) 3043. The index of synergy is calculated as:

$$\frac{dose of A}{{A}_{E}} + \frac{dose of B}{{B}_{E}} + \frac{( dose of A ) \times ( dose of B )}{{A}_{E} \times {B}_{E}}$$

in which the doses of the compounds A and B represent those used in a particular combination, and A_{E} and B_{E} are the individual doses of A and B respectively giving the same effect. If the result is less than 1, there is synergy, if the result is 1, the effect is additive; if the result is greater than 1, A and B are antagonistic. By plotting an isobologram of dose of A/A_{E} vs. dose of B/B_{E} the combination of maximum synergy can be determined. The synergistic ratio expressed in terms of the ratio by weight of the two compositions at synergistic amounts along the isobologram, especially at or near the point of maximum synergy, can then be used to determine formulations containing an optimally synergistic ratio of the two compounds.

Activity may e.g. be determined in known assay models for testing the activity of the individual components of the compositions.

The invention also provides products and methods for co-administration of a macrolide T-cell immunomodulator or immunosuppressant, e.g. 33-epichloro-33-desoxy-ascomycin or 5,6-dehydroascomycin, and a ceramide, e.g. ceramide 3, PC-9S or linoleic acid, at synergistically effective dosages, e.g.:
a method of treatment or prevention of a dermatological or mucosal disease such as atopic or contact dermatitis or dry skin, asteatotic eczema or xerosis in a subject suffering from or at risk for such condition, comprising co-administering synergistically effective amounts of a composition of the invention;
the use of a macrolide T-cell immunomodulator or immunosuppressant in the manufacture of a medicament for co-administration in synergistically effective amounts with a ceramide;
the use of a ceramide in the manufacture of a medicament for co-administration in synergistically effective amounts with a macrolide T-cell immunomodulator or immuno suppressant;
a kit of parts comprising a macrolide T-cell immunomodulator or immunosuppressant and a ceramide in separate unit dosage forms, preferably wherein the unit dosage forms are suitable for administration of the component compounds in synergistically effective amounts, together with instruction for use, optionally with further means for facilitating compliance with the administration of the component compounds, e.g. a label or drawings;
the use of a macrolide T-cell immunomodulator or immunosuppressant in the manufacture of a pharmaceutical kit which is to be used for facilitating co-administration with a ceramide;
the use of a ceramide in the manufacture of a pharmaceutical kit which is to be used for facilitating co-administration with a macrolide T-cell immunomodulator or immunosuppressant;
a macrolide T-cell immunomodulator or immunosuppressant and a ceramide as a combined pharmaceutical preparation for simultaneous, separate or sequential use, preferably in synergistically effective amounts, e.g. for the treatment or prevention of a dermatological or mucosal disease such as atopic or contact dermatitis or dry skin, asteatotic eczema or xerosis;
a pharmaceutical composition comprising a macrolide T-cell immunomodulator or immunosuppressant in combination or association with a ceramide, e.g. in synergistically effective amounts, together with at least one pharmaceutically acceptable diluent or carrier, e.g. for use in treatment or prevention of a dermatological or mucosal disease such as atopic or contact dermatitis or dry skin, asteatotic eczema or xerosis; and
a process for the preparation of a composition of the invention comprising mixing a macrolide T-cell immunomodulator or immunosuppressant and a ceramide, in combination or association with at least one pharmaceutically acceptable diluent or carrier.

Synergistic activity may be determined e.g. in a rat model of essential acid-deficient diet-induced dermatitis, e.g. as described in G. Imokawa et al., J. Clin. Invest. 94 (1994) 89-96.

By “synergistically effective amounts” is meant an amount of macrolide T-cell immunomodulator or immunosuppressant and an amount of ceramide which are individually below their respective effective dosages for a relevant indication, but which are pharmaceutically active on co-administration, e.g. in a synergistic ratio, for example as calculated above. Furthermore, “synergistically effective amounts” may mean an amount of macrolide T-cell immunomodulator or immunosuppressant and an amount of ceramide which are individually equal to their respective effective dosages for a relevant indication, and which result in a more than additive effect.

The molar amount of macrolide T-cell immunomodulator or immunosuppressant present is from roughly similar to, to significantly less than the amount of ceramide, preferably half as much or less. Synergistic ratios of macrolide T-cell immunomodulator or immunosuppressant to ceramide by weight are thus suitably from about 10:1 to about 1:50, preferably from about 5:1 to about 1:20, most preferably from about 1:1 to about 1:15, e.g. about 1:12.

The compositions of the invention can be administered as a free combination, or can be formulated into a fixed combination, which greatly enhances the convenience for the patient.

Absolute dosages of the compounds will vary depending on a number of factors, e.g. the individual, the route of administration, the desired duration, the rate of release of the active agent and the nature and severity of the condition to be treated. For example, the amount of active agents required and the release rate thereof may be determined on the basis of known in vitro and in vivo techniques, determining how long a particular active agent concentration in the blood plasma remains at an acceptable level for a therapeutic effect.

For example, in prevention and treatment of a dermatological or mucosal disease such as atopic or contact dermatitis or dry skin, asteatotic eczema or xerosis, an initial dosage of about 2-3 times the maintenance dosage is suitably administered, followed by a daily dosage of about 2-3 times the maintenance dosage for a period of from one to two weeks, and subsequently the dose is gradually tapered down at a rate of about 5% per week to reach the maintenance dosage. In general, synergistically effective amounts of 33-epichloro-33-desoxyascomycin and ceramide, e.g. ceramide 3, PC-9S or linoleic acid, on oral administration for use in prevention and treatment of atopic or contact dermatitis or dry skin, asteatotic eczema or xerosis in larger animals, e.g. man, are amounts of 33-epichloro-33-desoxyascomycin of up to about 2 mg/kg/day, e.g. from about 0.01 mg/kg/day to about 2 mg/kg/day, preferably about 0.5 mg/kg/day, in combination or co-administration with amounts of ceramide, such as ceramide 3, PC-9S or linoleic acid of up to about 50 mg/kg/day, e.g. from about 0.25 mg/kg/day to about 50 mg/kg/day, preferably about 2.5 mg/kg/day, in a synergistic ratio, as described. Suitable unit dosage forms for oral co-administration of these compounds thus may contain on the order of from about 0.5 mg to about 100 mg, preferably about 3 mg to about 30 mg of 33-epichloro-33-desoxyascomycin, and from about 10 mg to about 3000 mg, preferably about 50 mg to about 500 mg of ceramide. The daily dosage for oral administration is preferably taken in a single dose, but may be spread out over two, three or four dosages per day. For i.v. administration, the effective dosage is lower than that required for oral administration, e.g. about one fifth the oral dosage.

By “co-administration” is meant administration of the components of the compositions of the invention together or at substantially the same time, e.g. within fifteen minutes or less, either in the same vehicle or in separate vehicles, so that upon oral administration, for example, both compounds are present simultaneously in the gastrointestinal tract. Preferably, the compounds are administered as a fixed combination.

The compositions of the invention include compositions suitable for administration by any conventional route, in particular compositions suitable for administration either enterally, for example, orally, e.g. in the form of solutions for drinking, tablets or capsules, or parenterally, e.g. in the form of injectable solutions or suspensions; or topically, e.g. for the treatment of inflammatory conditions of the skin or mucosae, e.g. in the form of a dermal cream, ointment, ear drops, mousse, shampoo, solution, lotion, gel, emulgel or like preparation, e.g. in a concentration of from about 0.1% to about 10% by weight of each component, especially in combination or association with penetration enhancing agents, as well as for application to the eye, e.g. in the form of an ocular cream, gel or eye-drop preparation, for treatment of inflammatory conditions of the lungs and airways, e.g. in the form of inhalable compositions, and for mucosal application, e.g. in the form of vaginal tablets.

The compositions of the invention are suitably emulsions, microemulsions, emulsion preconcentrates or microemulsion preconcentrates, or solid dispersions, especially water-in-oil microemulsion preconcentrates or oil-in-water microemulsions, comprising the macrolide T-cell immunomodulator or immunosuppressant and the ceramide in a synergistic ratio.

The compositions of the invention can be prepared in conventional manner, e.g. by mixing a macrolide T-cell immunomodulator or immunosuppressant and a ceramide, in combination or association with at least one pharmaceutically acceptable diluent or carrier.

The active agent components may be in free form or pharmaceutically acceptable salt form as appropriate.

While the present invention primarily contemplates combination or association of just two pharmaceutically active components, it does not exclude the presence of further active agents, e.g. one further active agent, as far as they do not contradict the purpose of the invention.

The following Example illustrates the invention. The compounds are in free, i.e. neutral or base form unless specified otherwise.

### EXAMPLE

### Cream

| Component | Amount (g) |
|---|---|
| 33-Epichloro-33-desoxyascomycin | 1.00 |
| ceramide-3 | 1.00 |
| triglycerides, medium chain | 15.00 |
| oleyl alcohol | 10.00 |
| sodium cetylstearyl sulfate | 1.00 |
| cetyl alcohol | 4.00 |
| stearyl alcohol | 4.00 |
| glyceryl monostearate | 2.00 |
| benzyl alcohol | 1.00 |
| propylene glycol | 5.00 |
| citric acid | 0.05 |
| sodium hydroxide | * |
| water | ad 100.0 |

* amount required to adjust pH to 5.5

The preparation follows the conventional manufacturing procedures for an emulsion. The ascomycin and the ceramide are added to the heated homogeneous oily phase which contains triglycerides medium chain, oleyl alcohol, sodium cetylstearyl sulfate, cetyl alcohol, stearyl alcohol and glyceryl monostearate. In parallel, the water phase containing benzyl alcohol, propylene glycol, citric acid and sodium hydroxide is heated at the same temperature as the oily phase. The oily phase is added to the water phase and homogeneisation is performed. The resultant cream is cooled to room temperature.

1. A pharmaceutical composition comprising a macrolide T-cell immunomodulator or immunosuppressant in combination or association with a ceramide, together with at least one pharmaceutically acceptable diluent or carrier.

2. A composition according to claim 1 comprising 33-epichloro-33-desoxyascomycin in combination or association with ceramide 3, PC-9S or linoleic acid.

3. A method of treatment of a dermatological or mucosal disease such as atopic or contact dermatitis or dry skin, asteatotic eczema or xerosis in a subject suffering from or at risk for such condition, comprising co-administering a synergistically effective amount of a composition according to claim 1.

4. A process for the preparation of a composition according to claim 1 comprising mixing a macrolide T-cell immunomodulator or immunosuppressant and a ceramide, in combination or association with at least one pharmaceutically acceptable diluent or carrier.

5. A kit of parts comprising a macrolide T-cell immunomodulator or immunosuppressant and a ceramide in separate unit dosage forms, together with instructions for use.
